(19) United States
(12) Patent Application Publication
(10) Pub. No.: US 2012/0034198 A1
(43) Pub. Date: Feb. 9, 2012

(54) CARRIERS FOR STORAGE AND DELIVERY OF BIOLOGICS

(75) Inventors: Matthew Ryan Garner, Amarillo, TX (US); Joseph Flint, Ithaca, NY (US)

(73) Assignee: MICROBIOS, INC., Houston, TX (US)

(21) Appl. No.: 13/198,682

(22) Filed: Aug. 4, 2011

Related U.S. Application Data
(60) Provisional application No. 61/370,734, filed on Aug. 4, 2010.

(51) Int. Cl. A61K 35/74; A61P 1/14; A61P 1/12; A61P 3/00

(52) U.S. Cl. 424/93.44; 424/93.45; 424/93.4

(57) ABSTRACT

Processes and formulations for the administration and storage of dried microorganisms are disclosed. Microbial based products containing prebiotic formulations for administration and for storage of microorganisms are disclosed. These prebiotic formulations can act both for administration into an animal or human subject and act as to preserve or store the dried microorganisms.

### PRIORITY

This application claims priority to U.S. Provisional Application 61/370734, filed on Aug. 4, 2011.

### FIELD

The present disclosure relates to methods and compositions for preserving and administration of microorganisms. More specifically, the disclosure relates to methods for preserving and administration of lactic acid producing microorganisms and formulations for using such microorganisms in the inhibition of pathogenic growth.

### BACKGROUND

Beneficial bacteria colonize the intestinal tracts of mammals and can promote the well being of the host. The consumption of exogenous bacteria, often referred to as probiotics, can elicit beneficial effects upon a host. In humans, these probiotic bacteria have been shown to reduce the severity and duration of rotaviral-induced diarrhea, alleviate lactose intolerance, and enhance gastrointestinal immune function (Roberfroid 2000).

It is believed that the consumption of foods rich in probiotic bacteria, including lactic acid bacteria and bifidobacteria, leads to colonization of the human gastrointestinal tract of humans and has been demonstrated to alter the levels of lactic acid producing bacteria in feces (Roberfroid 2000). The addition of probiotic microorganisms to animal feed can improve animal efficiency and health. Specific examples include increased weight gain-to-feed intake ratio (feed efficiency), improved average daily weight gain, improved milk yield, and improved milk composition by dairy cows as described by U.S. Pat. Nos. 5,529,793 and 5,534,271. The administration of probiotic organisms can also reduce the incidence of pathogenic organisms in cattle, as described by U.S. Pat. No. 7,063,836.

Probiotics may work by competitive exclusion in which live microbial cultures act antagonistically toward specific organisms to cause a decrease in the numbers of that organism as reported by U.S. Pat. No. 7,323,166. Mechanisms of competitive exclusion include production of antibacterial agents (bacteriocins), production of metabolites (organic acids and hydrogen peroxide), competition for nutrients, and competition for adhesion sites on the gut epithelial surface as reported in U.S. Pat. No. 7,323,166.

Researchers have demonstrated that the consumption of probiotics by animals used in food production can improve the efficiency of animal production. Propionic acid is important in ruminal and intestinal fermentations and is a precursor to blood glucose synthesis (Baldwin 1983). Several examples are available that demonstrate the positive impact of feeding propionic acid-producing organisms to cattle. For example, U.S. Pat. Nos. 5,529,793 and 5,534,271, 6,455,063 and 6,887,489 demonstrate beneficial effects of propionic acid-producing bacteria upon cattle growth. Lactic acid bacteria (LAB) can inhibit pathogens in various food sources (Brashears et al., 2003). Lactic acid producing and lactate utilizing bacteria may also be helpful in inhibiting pathogenic growth in animals and improving the production of dairy products. U.S. Pat. No. 7,063,836. Lactic acid producing and lactate utilizing bacteria are beneficial for the utilization of feedstuffs by ruminants (U.S. Pat. Nos. 5,529,793 and 5,534,271) and have been fed to cattle to improve animal performance (Brashears et al., 2003).

Lactobacillus genus includes the most prevalently administered probiotic bacteria (Flint and Angert 2005). Lactobacillus is a genus of more than 25 species of gram-positive, catalase-negative, non-sporulating, rod-shaped organisms (Heilig et al., 2002). Lactobacillus species ferment carbohydrates to form lactic acid as reported in U.S. Pat. No. 7,323,166. Lactobacillus species are generally anaerobic, non-motile, and do not reduce nitrate as reported in U.S. Pat. No. 7,323,166. Lactobacillus species are often used in the manufacture of food products including dairy products and other fermented foods as reported by Heilig et al., 2002 and U.S. Pat. No. 7,323,166. Lactobacillus species inhabit various locations including the gastrointestinal tracts of animals and intact and rotting plant material as reported by Heilig et al., 2002 and U.S. Pat. No. 7,323,166. Lactobacillus strains appear to be present in the gastrointestinal tract of approximately 70% of humans that consume a Western-like diet. Heilig et al., 2002. The number of Lactobacillus cells in neonates is approximately 10⁵ colony forming units (CFU) per gram CFU/g of feces. Heilig et al., 2002. The amount in infants of one month and older is higher, ranging from 10⁶ to 10⁸ CFU/g of feces. Heilig et al., 2002.

Another means of altering intestinal flora is through the consumption of prebiotics. Prebiotics are food ingredients that are not readily digestible by endogenous mammalian enzymes and that selectively stimulate the growth and activity of selected groups of intestinal microorganisms that confer beneficial effects upon their host (Gibson 1995). Typically, it is probiotic microorganism populations that benefit from the presence of prebiotic compounds. Prebiotics can consist of oligosaccharides and other small molecules that serve as metabolic substrates for growth of probiotic microorganisms. While many diverse microorganisms inhabit the intestinal tract of a host organism, prebiotic compounds are only utilized by the probiotic microorganisms and lead to selective enhancement of probiotic growth. For Example, U.S. Pat. No. 4,873,229 reports the use of galacto-oligosaccharides as a way to increase the weight of and reduce the occurrence of scours in calves. Additionally, U.S Pat. Nos. 4,902,674, 4,987,124, and 5,032,579 report that the administration of prebiotic compounds can be used to reduce pathogenic populations.

Since prebiotics can serve as a metabolic substrate for the growth of probiotics, it has been reasoned that simultaneous administration of both might lead to synergistic effects. For instance, inclusion of a prebiotic should improve the survival of a probiotic microorganism because it ensures the presence of a readily available substrate for that probiotic microorganism and reduces substrate competition for nutrients with other microorganisms (Collins 1999). A term that may be used to describe formulations that include both prebiotics and probiotics is “synbiotics”.

The application of microorganisms to feed-stuffs is gaining world-wide popularity. Certain direct-fed microbials (DFM) mode of action require cells to be viable to be beneficial to a host. Many DFM products available rapidly lose viable cells and often contain insufficient viable cell concentrations to elicit a positive impact upon the host.

Various factors affect the viability of bacteria. For use as a probiotic, a lactic acid producing bacteria needs to be able remain viable during processing and storage protocols such as centrifugation, filtration, fermentation, freeze drying or lyophilization in which the bacteria may be subjected to freezing, high pressure, and high temperature as reported in U.S. Pat. No. 7,323,166. However, without adequate preservation many bioactive materials comprising bacterial cultures are sensitive to degradation, loss of activity, and/or viability in aqueous solutions, particularly at ambient or higher temperatures. Accordingly, bioactive materials comprising bacterial cultures often require refrigeration or have short shelf lives under ambient conditions.

When bioactive materials comprising bacteria are dried, they are also preserved in a concentrated form. These concentrated materials often need to be diluted to be disseminated in an efficient manner and at an effective dose. The diluting materials, commonly referred to as carriers, may comprise many different types of materials depending upon the product that requires dilution. Common carriers for biological materials include: cellulose, sugar, glucose, lactose, whey powder, or rice hulls. However, such carriers may not provide protection against loss of viable cells when exposed to temperatures above freezing. Products are often exposed to temperatures above freezing during manufacture, processing, packaging, shipment, and storage of the products.

Other carriers pose problems for delivery and application of products. Some carriers are essentially insoluble in aqueous solutions, thus making it difficult to apply the product in field operations.

Additionally, many probiotic bacteria are administered to animals in order to control or reduce pathogenic bacterial populations. Some of the described carriers (e.g. lactose, glucose, or whey powder) are readily utilized by these pathogens as a nutrient source, thus removing nutrients and potentially exacerbating the problem. Thus, there is need for a novel carrier for the storage and delivery of bioactive materials, particularly dried microorganisms, that provides improved protection during storage above freezing temperatures as well as not contribute to the proliferation of potentially pathogenic organisms.

Various reports exist regarding the combined use of prebiotics and probiotics in animals. For example, U.S. Pat. No. 7,229,818 B2 reports a formulation to increase the shelf stability of probiotic bacteria involving a process to coat freeze-dried bacteria in sodium alginate or potassium alginate salts in a low moisture environment. U.S. Pat. No. 5,785,990 reports a feed fortifier for pre-ruminant calves comprised of animal plasma, vitamins, minerals, electrolytes, allicin, probiotic microorganisms, a protein ingredient and less than 2% fructooligosaccharides. U.S. Pat. No. 6,797,266 reports a probiotic formulation containing Lactobacillus casei strain KE01 and several other components including trehalose, maltodextrin, whey proteins, egg albumin, gelatin, milk proteins, oils, phyto-chemicals and 0 to 10% fructooligosaccharide. U.S. Pat. Nos. 6,524,574 and No. 6,841,149 report a probiotic formulation that includes two Enterococcus and two Saccharomyces strains to administer to animals that contain a carbohydrate that can be utilized by the Enterococcus strains including glucose, fructose, lactose, maltose, and sucrose. U.S. Pat. No. 7,101,565 reports a probiotic and prebiotic combination which may contain a number of different prebiotics such as mucopolysaccharides that is directly administered to a gastrointestinal tract via tube inserted down the esophagus. U.S. Pat. No. 6,960,341 reports a combination of two Bifidobacteria, vitamins, and 0.5 to 5.0 grams of fructooligosaccharides per dose. The invention described incorporating 0.5 to 5.0 grams, preferably 1 to 3 grams, of fructooligosaccharides for each dose and is intended as source of nutrients to augment the growth and activity of the provided Bifidobacteria. While they say that shelf-life studies were performed and the product will contain greater than 5×10⁹ live cells after 2 years of storage at 22° C. for the two Bifidobacteria, there is no description of using the fructooligosaccharides as carrier to sustain the viability nor mention of using the FOS to store and deliver other types bacteria. U.S. Pat. No. 6,783,780 reports a nutritional supplement for the treatment of gastrointestinal disorders which includes oligosaccharides such as β-(arabino)galactans, β-(arabino)xylans, β-glucans, β-glucomannans, β-galactomannans, α-arabans, and inulin. U.S. Pat. No. 6,241,983 reports a composition which may include Lactobacillus acidophilus, Bifidobacterium adolescentis, immunoglobulins, and dietary fiber. The dietary fiber may include fructooligosaccharides not more than 36% of the total weight of the formulations. U.S. Pat. No. 4,873,229 reports feeding of prebiotics without probiotics such as galactooligosaccharides to calves to prevent the occurrence of scours and to increase the rate of weight gain. U.S. Pat. No. 6,241,983 reports a human health supplement that contains a mixture of Lactobacillus acidophilus, Bifidobacterium adolescentis, fiber and immunoglobulin. U.S. Patent Pub. Nos. US 2007/0280910, US 2007/0280911, US 2007/0280912 each report a combination of Bacillus subtilis, Bacillus coagulans and Enterococcus faecium that can be used in conjunction with fructo-oligosaccharides to treat irritable bowel syndrome and autism.

Because the efficacy of probiotic products requires that the dried microorganisms be ingested by the animal in a viable state (live) for efficacy, new methods and formulations may be needed to safely store and deliver the microorganisms to the animal.

### SUMMARY

The present disclosure describes new formulations comprising at least one microorganism and a prebiotic.

In certain aspects of the disclosure, the prebiotic may be any prebiotic. In still further aspects of the disclosure the prebiotic may be fructo-oligosaccharides (FOS), inulins, isomalto-oligosaccharides, gentio-oligosaccharides, lactilol, lactosucrose, lactulose, xylosucrose, glycosylsucrose, pyrodextrins, soybean oligosaccharides, galacto-oligosaccharides (GOS), transgalactose-oligosaccharides, xylo-oligosaccharides (XOS), Malto-oligosaccharides (MOS), mannan such as guar gum, locust bean gum, konjacor xanthan gum, pentosan, beta-glucan, arabinan, galactan, palantinose-oligosaccharides, gluco-oligosaccharides, cyclo-inulo-oligosaccharides, chito-oligosaccharides, agaro-oligosaccharides, neo-oligosaccharides, garo-oligosaccharides, pectins and pectic polysaccharides, etc. In particular aspects of the disclosure the carrier formulation includes fructo-oligosaccharides.

In certain aspects of the disclosure, the formulation comprises about 30% or more by weight of a prebiotic, in other aspects of the disclosure, the formulation comprises, 35%, 40%, 45%, 50%, 55%, 60%, 65%, 70%, 75%, 80%, 85%, 90%, 95% or more of a prebiotic by weight.

In certain aspects of the disclosure, the formulation may be a lyophilized or freeze-dried formulation.

In certain aspects of the disclosure, the at least one species or strain of probiotic microorganism retains at least 40% viability when stored for 48 hours at 22° C. In such aspects, the formulation may retain at least 15% viability when stored for 48 hours at 37° C.

Certain aspects of the disclosure concern a formulation with a flow agent. In such aspects, the flow agent can be any flow agent such as calcium stearate or silicon dioxide. In particular embodiments, the flow agent is calcium stearate. In such aspects, the formulation may possess the qualities of having at the a least one species or strain of probiotic microorganism retaining at least 60% viability when stored for 48 hours at 22° C. In such aspects, the formulation having least one species or strain of probiotic microorganism may retain at least 50% viability when stored for 48 hours at 37° C.

In particular embodiments, the microorganism can be any live probiotic bacteria or bacterial cells or in certain instances simply called cells. For example, the bacteria can be Bacillus subtilis, Bacillus licheniformis, Bifidobacterium adolescentis, Bifidobacterium animalis, Bifidobacterium bifidum, Bifidobacterium infantis, Bifidobacterium longum, Bifidobacterium thermophilum, Lactobacillus acidophilus, Lactobacillus agilis, Lactobacillus alactosus, Lactobacillus alimentarius, Lactobacillus amylophilus, Lactobacillus amylovorans, Lactobacillus amylovorus, Lactobacillus animalis, Lactobacillus batatas, Lactobacillus bavaricus, Lactobacillus bifermentans, Lactobacillus bifidus, Lactobacillus brevis, Lactobacillus buchnerii, Lactobacillus bulgaricus, Lactobacillus catenaforme, Lactobacillus casei, Lactobacillus cellobiosus, Lactobacillus collinoides, Lactobacillus confusus, Lactobacillus coprophilus, Lactobacillus coryniformis, Lactobacillus corynoides, Lactobacillus crispatus, Lactobacillus curvatus, Lactobacillus delbrueckii, Lactobacillus desidiosus, Lactobacillus divergens, Lactobacillus enterii, Lactobacillus farciminis, Lactobacillus fermentum, Lactobacillus frigidus, Lactobacillus fructivorans, Lactobacillus fructosus, Lactobacillus gasseri, Lactobacillus halotolerans, Lactobacillus helveticus, Lactobacillus heterohiochii, Lactobacillus hilgardii, Lactobacillus hordniae, Lactobacillus inulinus, Lactobacillus jensenii, Lactobacillus jugurti, Lactobacillus kandleri, Lactobacillus kefir, Lactobacillus lactis, Lactobacillus leichmannii, Lactobacillus lindneri, Lactobacillus malefermentans, Lactobacillus mali, Lactobacillus maltaromicus, Lactobacillus minor, Lactobacillus minutus, Lactobacillus mobilis, Lactobacillus murinus, Lactobacillus pentosus, Lactobacillus plantarum, Lactobacillus pseudoplantarum, Lactobacillus reuteri, Lactobacillus rhamnosus, Lactobacillus rogosae, Lactobacillus tolerans, Lactobacillus torquens, Lactobacillus ruminis, Lactobacillus sake, Lactobacillus salivarius, Lactobacillus sanfrancisco, Lactobacillus sharpeae, Lactobacillus trichodes, Lactobacillus vaccinostercus, Lactobacillus viridescens, Lactobacillus vitulinus, Lactobacillus xylosus, Lactobacillus yamanashiensis, Lactobacillus zeae, Pediococcus acidlactici, Pediococcus pentosaceus, Streptococcus cremoris, Streptococcus discetylactis, Streptococcus faecium, Streptococcus intermedius, Streptococcus lactis, Streptococcus thermophilus, Enterococcus faecium, and combinations thereof. Furthermore, a lactic acid-producing microorganism can be a strain of Lactobacillus spp., such as the MRL1, M35, LA45, L411, NPC747, NPC750, D3, and L7 strains.

In certain aspects of the disclosure, the probiotic microorganism is a lactic-acid producing microorganism. In still further aspects of the invention, the probiotic microorganism is Lactobacillus acidophilus, Lactobacillus agilis, Lactobacillus alactosus, Lactobacillus alimentarius, Lactobacillus amylophilus, Lactobacillus amylovorans, Lactobacillus amylovorus, Lactobacillus animalis, Lactobacillus batatas, Lactobacillus bavaricus, Lactobacillus bifermentans, Lactobacillus bifidus, Lactobacillus brevis, Lactobacillus buchnerii, Lactobacillus bulgaricus, Lactobacillus catenaforme, Lactobacillus casei, Lactobacillus cellobiosus, Lactobacillus collinoides, Lactobacillus confusus, Lactobacillus coprophilus, Lactobacillus coryniformis, Lactobacillus corynoides, Lactobacillus crispatus, Lactobacillus curvatus, Lactobacillus delbrueckii, Lactobacillus desidiosus, Lactobacillus divergens, Lactobacillus enterii, Lactobacillus farciminis, Lactobacillus fermentum, Lactobacillus frigidus, Lactobacillus fructivorans, Lactobacillus fructosus, Lactobacillus gasseri, Lactobacillus halotolerans, Lactobacillus helveticus, Lactobacillus heterohiochii, Lactobacillus hilgardii, Lactobacillus hordniae, Lactobacillus inulinus, Lactobacillus jensenii, Lactobacillus jugurti, Lactobacillus kandleri, Lactobacillus kefir, Lactobacillus lactis, Lactobacillus leichmannii, Lactobacillus lindneri, Lactobacillus malefermentans, Lactobacillus mali, Lactobacillus maltaromicus, Lactobacillus minor, Lactobacillus minutus, Lactobacillus mobilis, Lactobacillus murinus, Lactobacillus pentosus, Lactobacillus plantarum, Lactobacillus pseudoplantarum, Lactobacillus reuteri, Lactobacillus rhamnosus, Lactobacillus rogosae, Lactobacillus tolerans, Lactobacillus torquens, Lactobacillus ruminis, Lactobacillus sake, Lactobacillus salivarius, Lactobacillus sanfrancisco, Lactobacillus sharpeae, Lactobacillus trichodes, Lactobacillus vaccinostercus, Lactobacillus viridescens, Lactobacillus vitulinus, Lactobacillus xylosus, Lactobacillus yamanashiensis, Lactobacillus zeae, Lactobacillus strain MRL1, Lactobacillus strain M35, Lactobacillus strain LA45, Lactobacillus strain LA51, Lactobacillus strain L411, Lactobacillus strain NPC 747, Lactobacillus strain NPC 750, Lactobacillus strain D3, Lactobacillus strain L7, Lactobacillus strain DF7, Lactobacillus strain MB1 or a combination thereof.

In certain aspects of the disclosure, the probiotic microorganism is a propionic acid-producing microorganism. In still further aspects, the propionic acid-producing microorganism is Propionibacterium strain PF24, P5, P63, P1, or MRP1 or a combination thereof.

Consistent with long standing patent law, the words “a” and “an” denote “one or more,” when used in the text or claims of this specification in conjunction with the word “comprising” or where the context of the usage suggests that, from either a grammatical or scientific standpoint, these words should so denote.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Graph showing improved viability of dried bacteria in a carrier agent when exposed to room temperature (22° C.).

FIG. 2. Graph showing improved viability of dried bacteria in a carrier agent when exposed to 37° C.

FIG. 3. Graph showing the growth of Lactobacillus and other pathogenic bacteria using lactose as a carbon source.

FIG. 4. Graph showing the final pH values of the growth medium after growth of bacteria using lactose as a carbon source.

FIG. 5. Graph showing the growth of Lactobacillus and other pathogenic bacteria using fructooligosaccharides as a carbon source.

FIG. 6. Graph showing the final pH values of the growth medium after growth of bacteria using fructooligosaccharides as a carbon source.

FIG. 7. Graph showing reduction of E. coli numbers when incubated for 6 hours with Lactobacillus using glucose as a carbon source.

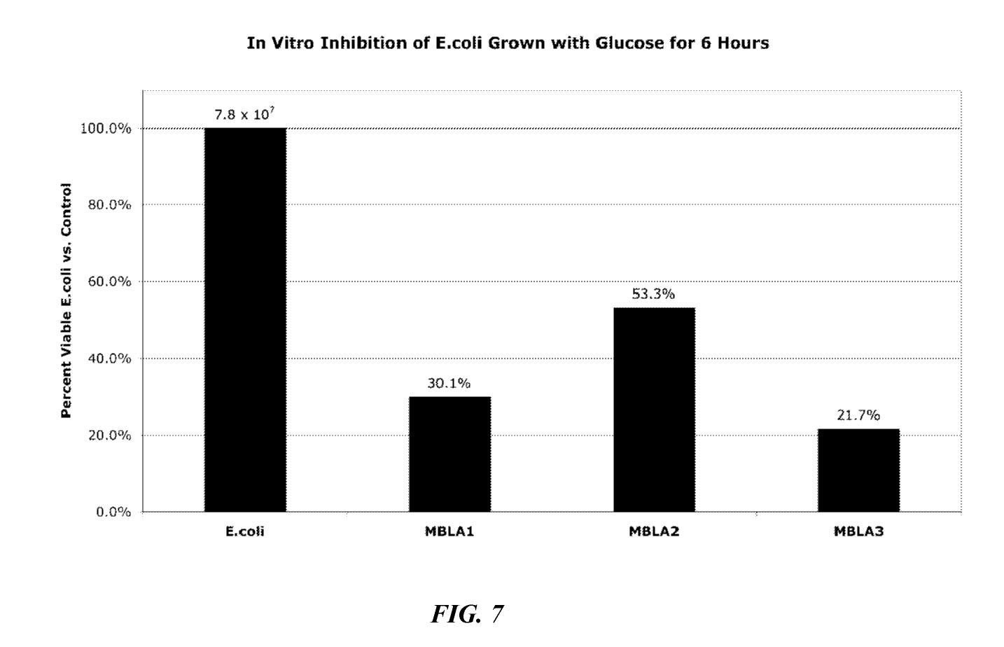

FIG. 8. Graph showing reduction of E. coli numbers when incubated for 6 hours with Lactobacillus using fructooligosaccharides as a carbon source.

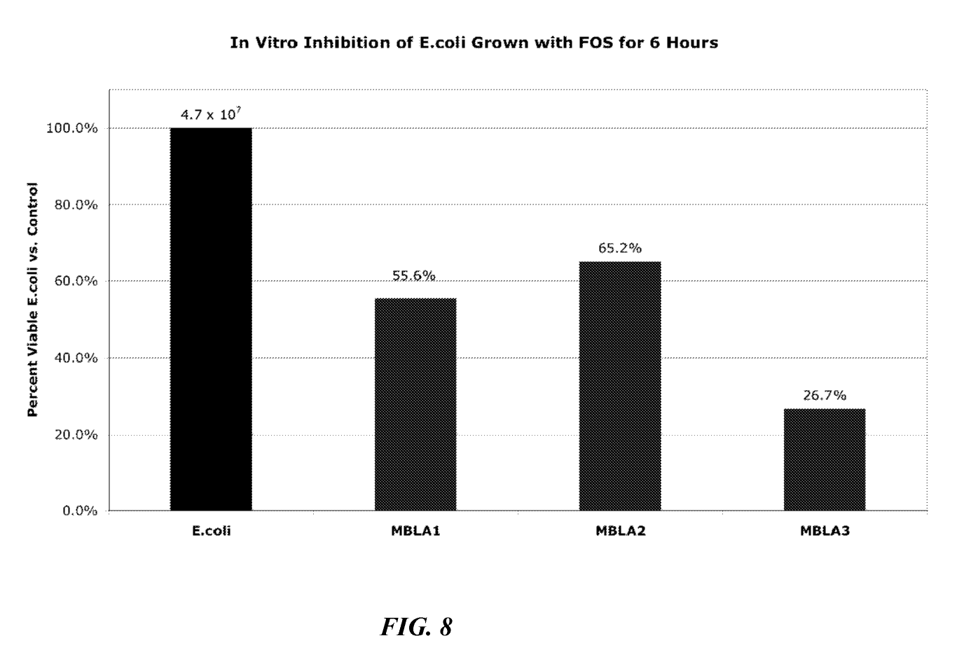

FIG. 9. Graph showing reduction of E. coli numbers when incubated for 12 hours with Lactobacillus using fructooligosaccharides as a carbon source.

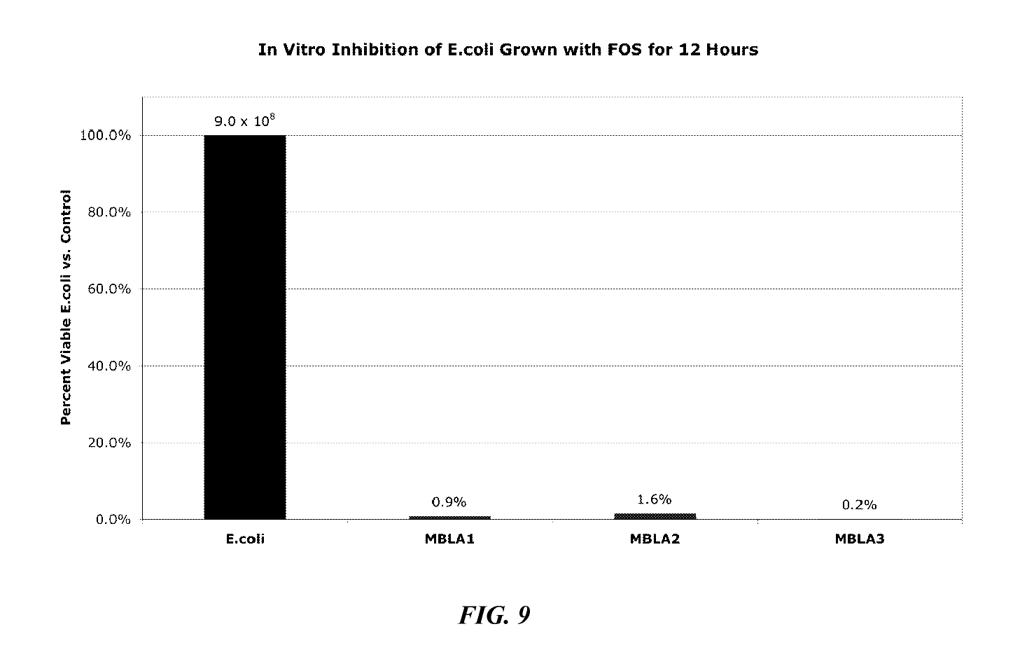

### DETAILED DESCRIPTION

### a. Terminology

In this specification and the claims that follow, reference will be made to a number of terms which may be considered to have the following meanings:

By “reduce” or other forms of the word, such as “reducing” or “reduction,” may in certain instances refer to lowering of an event or characteristic (e.g., microorganism growth or survival). It is understood that this is typically in relation to some standard or expected value, in other words it is relative, but that it is not always necessary for the standard or relative value to be referred to. For example, “reduces the population of bacteria” in certain instances may refer to lowering the amount of bacteria relative to a standard or a control.

By “treat” or other forms of the word, such as “treated” or “treatment,” may, in certain instances mean to administer a composition or to perform a method in order to reduce, prevent, inhibit, break-down, or eliminate a particular characteristic or event (e.g., microorganism growth or survival).

The term “viable cell” may in certain instances mean a microorganism that is alive and capable of regeneration and/or propagation, while in a vegetative, frozen, preserved, or reconstituted state.

The term “viable cell yield” or “viable cell concentration” may, in certain instances refer to the number of viable cells in a liquid culture, concentrated, or preserved state per a unit of measure, such as liter, milliliter, kilogram, gram or milligram.

The term “cell preservation” in certain instances may refer to a process that takes a vegetative cell and preserves it in a metabolically inert state that retains viability over time. As used herein, the term “product” in certain instances may refer to a microbial composition that can be blended with other components and contains specified concentration of viable cells that can be sold and used.

The terms “microorganism” or “microbe” in certain instances may refer to an organism of microscopic size, to a single-celled organism, and/or to any virus particle. Our definition of microorganism includes Bacteria, Archaea, single-celled Eukaryotes (protozoa, fungi, and ciliates), and viral agents. The term “microbial” in certain instances may refer to processes or compositions of microorganisms, thus a “microbial-based product” is a composition that includes microorganisms, cellular components of the microorganisms, and/or metabolites produced by the microorganisms. Microorganisms can exist in various states and occur in vegetative, dormant, or spore states. Microorganisms can also occur as either motile or non-motile, and may be found as planktonic cells (unattached), substrate affixed cells, cells within colonies, or cells within a biofilm.

The term “prebiotic” in certain instances may refer to food ingredients that are not readily digestible by endogenous host enzymes and confer beneficial effects on an organism that consumes them by selectively stimulating the growth and/or activity of a limited range of beneficial microorganisms that are associated with the intestinal tract.

The term “probiotic” in certain instances may refer to one or more live microorganisms that confer beneficial effects on a host organism. Benefits derived from the establishment of probiotic microorganisms within the digestive tract include reduction of pathogen load, improved microbial fermentation patterns, improved nutrient absorption, improved immune function, aided digestion and relief of symptoms of irritable bowel disease and colitis.

The term “synbiotic” in certain instances may refer to a composition that contains both probiotics and prebiotics. Synbiotic compositions are those in which the prebiotic compound selectively favors the probiotic microorganism.

The term “gastrointestinal tract” in certain instances may refer to the complete system of organs and regions that are involved with ingestion, digestion, and excretion of food and liquids. This system generally consists of, but not limited to, the mouth, esophagus, stomach and or rumen, intestines (both small and large), cecum (plural ceca), fermentation sacs, and the anus.

The term “pathogen” in certain instances may refer to any microorganism that produces a harmful effect and/or disease state in a human or animal host.

The term “fermentation” in certain instances may refer to a metabolic process performed by an organism that converts one substrate to another in which the cell is able to obtain cellular energy, such as when an organism utilizes glucose and converts it to lactic acid or propionic acid. Many of the end-substrates formed in fermentation processes are volatile fatty acids.

The term “volatile fatty acids” in certain instances may refer to short-chain fatty acids containing six or fewer carbon atoms and at least one carboxyl group. Some examples of VFAs include, but are not limited to: lactic acid, acetic acid, propionic acid, butyric acid, isobutyric acid, valeric acid, and isovaleric acid, which are products of microbial fermentation within the digestive tracts of animals. Volatile fatty acids can be absorbed through the intestines of animals and used as an energy or carbon source. Microbes produce VFAs based on available substrates and also rely upon VFAs for energy and carbon sources.

The term “lactic acid” in certain instances may refer to a byproduct of glucose fermentation resulting in a three-carbon acid with the chemical formula C₃H₆O₃. This includes, but is not limited to, lactic acid derived from specific strains of bacteria or lactic acid derived from other types of organisms. Lactic acid can be microbialstatic, microbialcidal, bacteriostatic, bacteriocidal or bacteriolytic; these concepts are known to skilled persons. “Lactic acid producing” refers to any organism that generates lactic acid.

The term “bacteriocin(s)” in certain instances may refer to (poly) peptides and proteins that inhibit one or more microbial species. This includes, but is not limited to, (poly) peptides or proteins that were derived from specific strains of bacteria or (poly) peptides that are derived from other types of organisms. The bacteriocin can be microbialstatic, microbialcidal, bacteriostatic, bacteriocidal, or bacteriolytic; these concepts are known to skilled persons. For the treatment of produce and other food products the bacteriocin is preferably microbialcidal or bacteriocidal. “Bacteriocin-producing” in certain instances may refer to any organism that generates bacteriocins.

### b. Introduction

The present disclosure pertains to a novel carrier formulation including prebiotic compounds used for the storage and delivery of dried microorganisms. The formulation can be adjusted to provide beneficial effects to many types of animals, including ruminal fermentors, cecal fermentors and intestinal fermentors.

In one aspect of the disclosure, the formulation may in certain instances be fed to ruminal fermentors to reduce scours events and improve animal health and performance. Ruminal fermentors may include, but are not limited to: cattle, sheep, goats, camels, llama, bison, buffalo, deer, wildebeest, antelope, and any other pre-gastric fermentor. In another aspect of the disclosure, the formulation may in certain instances be fed to cecal fermentors to reduce scours events and improve animal health and performance. Cecal fermentors that might benefit from said invention include but are not limited to: horses, ponies, elephants, rabbits, hamsters, rats, hyraxes, guinea pigs, and any other post-gastric fermentor that using the cecum as the primary location of fermentative digestion. In another aspect of the invention, the formulation is fed to intestinal fermentors to reduce scours events and improve animal health and performance. Intestinal fermentors that might benefit from said invention include but are not limited to: humans, pigs, chickens, and other post-gastric fermentor using the large intestine as the primary location of fermentative digestion. In each case, the composition is packaged in format that ensures survival of the probiotic into the gastrointestinal system of the animal.

### c. Microorganisms

Certain aspects of the disclosure include the application of one or more viable micro-organisms to the aforementioned formulation for animal feed. The microorganisms can be different microorganisms and/or different strains. For example, one, two, three, four, five, six, and so on different microorganisms and/or strains can be applied. The application of multiple types of different microorganisms and/or different strains can lead to synergistic effects.

It is preferred that the probiotic mixture could contain any number of microorganisms and or microbial components, spores, and/or metabolites. Examples of bacterial species that could be used for the probiotic mixture include but are not limited to the group consisting of: Enterococcus faecium, Bacillus licheniformis, Lactococcus lactis, Bacillus subtilis, Bifidobacterium adolescentis, Bifidobacterium animalis, Bifidobacterium bifidum, Bifidobacterium infantis, Bifidobacterium longum, Bifidobacterium thermophilum, Lactobacillus acidophilus, Lactobacillus agilis, Lactobacillus alactosus, Lactobacillus alimentarius, Lactobacillus amylophilus, Lactobacillus amylovorans, Lactobacillus amylovorus, Lactobacillus animalis, Lactobacillus batatas, Lactobacillus bavaricus, Lactobacillus bifermentans, Lactobacillus bifidus, Lactobacillus brevis, Lactobacillus buchnerii, Lactobacillus bulgaricus, Lactobacillus catenaforme, Lactobacillus casei, Lactobacillus cellobiosus, Lactobacillus collinoides, Lactobacillus confusus, Lactobacillus coprophilus, Lactobacillus coryniformis, Lactobacillus corynoides, Lactobacillus crispatus, Lactobacillus curvatus, Lactobacillus delbrueckii, Lactobacillus desidiosus, Lactobacillus divergens, Lactobacillus enterii, Lactobacillus farciminis, Lactobacillus fermentum, Lactobacillus frigidus, Lactobacillus fructivorans, Lactobacillus fructosus, Lactobacillus gasseri, Lactobacillus halotolerans, Lactobacillus helveticus, Lactobacillus heterohiochii, Lactobacillus hilgardii, Lactobacillus hordniae, Lactobacillus inulinus, Lactobacillus jensenii, Lactobacillus jugurti, Lactobacillus kandleri, Lactobacillus kefir, Lactobacillus lactis, Lactobacillus leichmannii, Lactobacillus lindneri, Lactobacillus malefermentans, Lactobacillus mali, Lactobacillus maltaromicus, Lactobacillus minor, Lactobacillus minutus, Lactobacillus mobilis, Lactobacillus murinus, Lactobacillus mucosae, Lactobacillus pentosus, Lactobacillus plantarum, Lactobacillus pseudoplantarum, Lactobacillus reuteri, Lactobacillus rhamnosus, Lactobacillus rogosae, Lactobacillus tolerans, Lactobacillus torquens, Lactobacillus ruminis, Lactobacillus sake, Lactobacillus salivarius, Lactobacillus sanfrancisco, Lactobacillus sharpeae, Lactobacillus trichodes, Lactobacillus vaccinostercus, Lactobacillus viridescens, Lactobacillus vitulinus, Lactobacillus xylosus, Lactobacillus yamanashiensis, Lactobacillus zeae, Pediococcus acidilactici, Pediococcus pentosaceus, Streptococcus cremoris, Streptococcus diacetylactis, Streptococcus faecium, Streptococcus intermedius, Streptococcus lactis, Streptococcus thermophilus, Propionibacterium spp., Propionibacterium freudenreichii, Propionibacterium acidipropionici, Propionibacterium jensenii, Propionibacterium thoenii, Propionibacterium cyclohexanicum, Propionibacterium granulosum, Propionibacterium microaerophilum, Propionibacterium propionicum, Propionibacterium acnes, Propionibacterium australiense, Propionibacterium avidum and strains and/or combinations thereof. Furthermore, a lactic acid-producing microorganism can be a strain of Lactobacillus spp., such as the MRL1, M35, LA45, LA51, L411, NPC 747, NPC 750, D3, and L7 strains. Examples of a lactic acid-utilizing and/or propionic acid-producing organism include the Propionibacterium spp. strains PF24, P5, P63, P1, and MRP1. The novel carrier can also be used to deliver other microorganisms such as yeasts, Saccharomyces cerevisae, Saccharomyces bayanus, Saccharomyces boulardii, Saccharomyces cariocanus, molds, Aspergillus oryzae, Aspergillus niger, Aspergillus amylovorus, Aspergillus flavus, Furthermore, methanogenic Archaea including, but not limited to, Methanobacterium formicicum, Methanobacterium aarhusense, Methanobacterium alcaliphilum, Methanobacterium beijingense, Methanobacterium bryantii, Methanobacterium congolense, Methanobacterium espanolae, Methanobacterium ivanovii, Methanobacterium oryzae, Methanobacterium palustre, Methanobacterium subterraneum, Methanobacterium thermaggregans, Methanobacterium uliginosum, Methanobrevibacter ruminantium, Methanobrevibacter acididurans, Methanobrevibacter arboriphilus, Methanobrevibacter curvatus, Methanobrevibacter cuticularis, Methanobrevibacter filiformis, Methanobrevibacter gottschalkii, Methanobrevibacter oxalis, Methanobrevibacter smithii, Methanobrevibacter thaueri, Methanobrevibacter woesei, Methanobrevibacter wolinii, Methanosphaera stadtmanae, Methanosphaera cuniculi, Methanothermobacter thermautotrophicus, Methanobacterium thermoalcaliphilum, Methanobacterium thermoformicicum, Methanothermobacter defluvii, Methanothermobacter marburgensis, Methanothermobacter thermoflexus, Methanothermobacter thermophilus, Methanothermobacter wolfeii, Methanothermus fervidus, Methanothermus sociabilis, Methanococcus vannielii, Methanococcus aeolicus, Methanococcus maripaludis, Methanococcus deltae, Methanococcus voltae, Methanothermococcus thermolithotrophicus, Methanothermococcus okinawensis, Methanocaldoccocus jannaschii, Methanocaldoccocus fervens, Methanocaldoccocus indicus, Methanocaldoccocus infernus, Methanocaldoccocus vulcanius, Methanotorris igneus, Methanotorris formicicus, Methanomicrobium mobile, Methanoculleus bourgensis, Methanoculleus olentangyi, Methanoculleus oldenburgensis, Methanoculleus chikugoensis, Methanoculleus marisnigri, Methanoculleus palmolei, Methanoculleus thermophilus, Methanoculleus submarinus, Methanofollis tationis, Methanofollis aquaemaris, Methanofollis formosanus, Methanofollis liminatans, Methanogenium cariaci, Methanogenium frigidum, Methanogenium frittonii, Methanogenium marinum, Methanogenium organophilum, Methanolacinia paynteri, Methanoplanus limicola, Methanoplanus endosymbiosus, Methanoplanus petrolearius, Methanocorpusculum parvum, Methanocorpusculum aggregans, Methanocorpusculum bavaricum, Methanocorpusculum labreanum, Methanocorpusculum sinense, Methanocalculus halotolerans, Methanocalculus chunghsingensis, Methanocalculus pumilus, Methanocalculus taiwanensis, Methanospirillum hungatei, Methanosarcina barkeri, Methanosarcina acetivorans, Methanosarcina baltica, Methanosarcina lacustris, Methanosarcina mazei, Methanosarcina frisia, Methanosarcina semesiae, Methanosarcina siciliae, Methanosarcina thermophila, Methanosarcina vacuolata, Methanococcoides methylutens, Methanococcoides burtonii, Methanohalobium evestigatum, Methanohalophilus mahii, Methanohalophilus halophilus, Methanohalophilus portucalensis, Methanolobus tindarius, Methanolobus bombayensis, Methanolobus oregonensis, Methanolobus taylorii, Methanolobus vulcani, Methanosalsum zhilinae, Methanomethylovorans hollandica, Methanimicrococcus blatticola, Methanosaeta concilii, Methanosaeta thermophila, etc.

The amount of microorganism contained in the carrier can be any amount sufficient to achieve the desired effects on the animal. This amount can be anywhere from 1 to 10¹² organisms per gram of carrier. For example, amounts of about 10⁴ cfu/gram carrier, about 5×10⁴ cfu/gram carrier, about 10⁵ cfu/gram carrier, about 5×10⁵ cfu/gram carrier, about 1×10⁷ cfu/gram carrier, about 1×10⁸ cfu/gram carrier, 1×10⁹ cfu/gram carrier, 1×10¹⁰ cfu/gram carrier, 1×10¹¹ cfu/gram carrier, or ranges between 1 to 10¹² organisms per gram of carrier can be used.

### d. Carriers And Formulations

In certain aspects of the disclosure the carrier formulation may include one or more prebiotic compounds. Examples of prebiotic compounds that used in the formulation include but are not limited to the following: fructo-oligosaccharides (FOS), inulins, isomalto-oligosaccharides, gentio-oligosaccharides, lactilol, lactosucrose, lactulose, xylosucrose, glycosylsucrose, pyrodextrins, soybean oligosaccharides, galacto-oligosaccharides (GOS), transgalactose-oligosaccharides, xylo-oligosaccharides (XOS), Malto-oligosaccharides (MOS), mannan such as guar gum, locust bean gum, konjacor xanthan gum, pentosan, beta-glucan, arabinan, galactan, palantinose-oligosaccharides, gluco-oligosaccharides, cyclo-inulo-oligosaccharides, chito-oligosaccharides, agaro-oligosaccharides, neo-oligosaccharides, garo-oligosaccharides, pectins and pectic polysaccharides, etc. In particular aspects of the disclosure the carrier formulation includes fructo-oligosaccharides.

In certain aspects of the disclosure, the carrier may be any number of different percentages (weight per weight, weight per volume, or volume per volume) of the final product. The carrier can comprise any amount of about 99.9%, about 95%, about 90%, about 80%, about 70%, about 60% about 50%, about 40%, about 30% and so on. The remaining composition can also include other carriers such as lactose, glucose, sucrose, salt, cellulose, whey protein, etc. In specific aspects of the disclosure, the carrier may be 50% or more of the total product.

In certain aspects of the disclosure, the carrier and composition can also have defined properties, such as solubility/insolubility in water or solubility/insolubility in fat, etc.

In certain aspects of the disclosure, other chemicals or materials principally used for the reduction or absorption of moisture may also be included. These may include, but are not limited to: calcium stearate, sodium aluminosilicate, silica, calcium carbonate, zeolite, bicarbonates, sodium sulfate, silicon dioxide, or ascorbic acid.

In certain aspects of the disclosure, other chemicals or materials principally used for the reduction or absorption of oxygen may also be included. These may include, but are not limited to, iron oxides, ascorbic acid, sodium sulfide, and silica materials.

In certain aspects of the disclosure, the biological material mixed with the present carrier can be stored in a pouch or bag fabricated from various materials, a bottle fabricated from a variety of materials, a capsule, a box, or other storage container.

In certain aspects of the disclosure, the biological material mixed in the present carrier may also be used for the application onto a variety of foods including, but not limited to, meats, vegetables, fruits, processed foods, or others.

In certain aspects of the present disclosure wherein formulations are contemplated for preservation, such preservation may include a process of freezing, lyophilization, freeze-drying and/or spray-drying. In certain aspects, the preserved bacteria contain a viable cell concentration of 1×10⁸ to 5×10¹² cfu/g. Still further, in certain aspects the concentrations range from 5×10¹⁰ cfu/g to 5×10¹³ cfu/g of bacteria.

Flow agents may also be included in the carrier formulations. In certain aspects, the flow agent may be calcium stearate, silica, silicon dioxide and the like. In preferred aspects the flow agent is calcium stearate. In specific aspects, the flow agent is calcium stearate and is combined with in a carrier formulation of fructo-oligosaccharide in an amount of 2% by weight or 2% by volume. This particular formulation is Fos-flow. In other aspects, the flow agent may comprise about 1 to about 50% of the carrier formulation or some number within this range.

### e. Preservation Matrices

In certain instances, a bacterial formulation for administration to a subject or a surface or other target can include a preservation matrix, which contains and preserves the bacterial culture. Such a matrix may include a biologically active binding agent, an antioxidant, a polyol, a carbohydrate and a proteinaceous material. For example, the matrix may have a pH of from about 5.0 to about 7.0. Such a preservation matrix may be capable of maintaining at least about 10⁶ viable cells for a period of at least about 12 months in vitro. In other examples, such a matrix maintains at least about 10⁷ viable cells for a period of at least about 12 months in vitro, and more preferably, at least about 10⁸ viable cells for a period of at least about 12 months in vitro. A preservation matrix may be comprised of ingredients to minimize the damaging effects encountered during the preservation process and to provide functional properties. For example when a Lactobacillus strain of the present invention is added to a preservation matrix for preservation, it is may converted from an actively growing metabolic state to a metabolically inactive state. In formulations of the present invention wherein a preservation matrix is contemplated, a biologically acceptable binding agent can be used to both affix the bacterial culture or cultures to an inert carrier during a preservative process and to provide protective effects (i.e., maintains cell viability) throughout preservation and storage of the microbial cells. Preferred biologically acceptable binding agents for use in a preservation matrix include, but are not limited to a water-soluble gum, carboxymethyl cellulose and/or gelatin. A biologically acceptable binding agent typically comprises from about 10% to about 20% by weight of the preservation matrix, and preferably comprises about 14% by weight of the preservation matrix. In one embodiment, a preservation matrix of the present invention comprises about 14% gelatin by weight of the preservation matrix.

Antioxidants included in a preservation matrix may be provided to retard oxidative damage to the microbial cells during the preservation and storage process. A particularly preferred antioxidant is sodium ascorbate. An antioxidant typically comprises from about 0.1% to about 1.0% by weight of the preservation matrix, and preferably comprises about 0.5% by weight of the preservation matrix. In one embodiment, a preservation matrix of the present invention comprises about 0.5% sodium ascorbate by weight of the preservation matrix.

Polyols (i.e., polyhydric alcohols) included in a preservation matrix may be provided to maintain the native, uncollapsed state of cellular proteins and membranes during the preservation and storage process. In particular, polyols interact with the cell membrane and provide support during the dehydration portion of the preservation process. Preferred polyols include, but are not limited to xylitol, adonitol, glycerol, dulcitol, inositol, mannitol, sorbitol and/or arabitol. A polyol typically comprises from about 1% to about 25% by weight of the preservation matrix, and preferably comprises about 6% by weight of the preservation matrix. In one embodiment, a preservation matrix of the present invention comprises about 6% xylitol by weight of the preservation matrix.

Carbohydrates included in a preservation matrix may be provided to maintain the native, uncollapsed state of cellular proteins and membranes during the preservation and storage process. In particular, carbohydrates provide cell wall integrity during the dehydration portion of the preservation process. Preferred carbohydrates include, but are not limited to dextrose, lactose, maltose, sucrose, fructose and/or any other monosaccharide, disaccharide or polysaccharide. A carbohydrate typically comprises from about 0.5% to about 5% by weight of the preservation matrix, and preferably comprises about 2.5% by weight of the preservation matrix. In one embodiment, a preservation matrix of the present invention comprises about 2.5% dextrose by weight of the preservation matrix.

A proteinaceous material included in a preservation matrix may provide further protection of the microbial cell during the dehydration portion of the preservation process. Preferred proteinaceous materials include, but are not limited to skim milk and albumin. A proteinaceous material typically comprises from about 0.5% to about 5% by weight of the preservation matrix, and preferably comprises about 1.5% by weight of the preservation matrix. In one embodiment, a preservation matrix of the present invention comprises about 1.5% skim milk by weight of the preservation matrix.

One example of a method of preserving microbial cells within a preservation matrix includes coating the cell matrix suspension onto an inert carrier that preferably is a maltodextrin bead. The coated beads can then be dried, preferably by a fluid bed drying method. Fluid bed drying methods are well known in the art. For example, maltodextrin beads may be placed into a fluid bed dryer and dried at 33° C. The air pressure may be set to 1 bar, the cell suspension matrix can then be sprayed onto the beads and the heat is increased to 38° C. The coated beads are then allowed to dry for an additional period of time. The coated maltodextrin beads can be stored as a powder, placed into gelatin capsules, or pressed into tablets.

In other formulations of the disclosure, the single strains or combinations of strains of bacteria contemplated to be cultured can be formulated as a hard gelatin capsule. Gelatin capsules are commercially available and are well known in the art. In this embodiment, the above preservation method further comprises dispensing the cell suspension matrix to a gelatin capsule, chilling the gelatin capsule until the cell suspension matrix forms a non-fluid matrix and to affix the gel to the interior wall of the gelatin capsule, and desiccating the gelatin capsule in a desiccation chamber. The step of dispensing can be accomplished by any means known in the art, and includes manual, semi-automated and automated mechanisms. The chilling step is performed at from about 4° C. to about 6° C. The step of desiccating the gelatin capsule can include the steps of (i) providing dry air to the desiccation chamber containing less than about 25% moisture, at a temperature from about 24° C. to about 32° C.; and (ii) removing humidified air from the desiccation chamber.

In this formulation of the present invention the desiccation process may proceed for about 1 to about 6 hours. The desiccation chamber can include a compressor, at least one hydrocarbon scrubbing filter and a chilled air compressor with or without a desiccant silica gel (or any other suitable desiccant material) column, in series. The air entering the chamber (dry air) preferably contains less than about 25% moisture, and more preferably less than about 15% moisture, and even more preferably less than about 5% moisture, down to as little as zero moisture. The dry air should preferably have a temperature from about 24° C. to about 32° C. This method allows preservation of microbial cells in a controlled environment with room temperature air in a short period of time. Further examples of embodiments of preservation matrices and gelatin capsule formulations may be found in U.S. Pat. No. 6,468,526 which is herein incorporated by reference in its entirety.

### f. Microencapsulation

In certain applications, the bacteria cultured with the methods described herein may be placed in a microencapsulation formulation. Such microencapsulation formulations may have applicability for example in administration to subjects via oral, nasal, rectal, vaginal or urethral routes. Spray drying is the most commonly used microencapsulation method in the food industry, is economical and flexible, and produces a good quality product. The process involves the dispersion of the core material into a polymer solution, forming an emulsion or dispersion, followed by homogenisation of the liquid, then atomisation of the mixture into the drying chamber. This leads to evaporation of the solvent (water) and hence the formation of matrix type microcapsules.

For example O'Riordan et al., 2001 reported microencapsulation and spray drying of Bifidobacterium cells with a spray inlet temperature of 100° C. and low outlet temperature of 45° C. The cells were reported to be encapsulated satisfactorily to produce micro spheres with gelatinized modified starch as a coating material (O'Riordan et al., 2001). In this study, spray drying was found to be a valuable process for encapsulating Bifidobacteria. The process of spray drying is economical, easily scaled up and uses equipment readily available in the food industry (Gibbs et al., 1999). A previous report indicated that survival of probiotic bacteria during spray drying decreased with increasing inlet temperatures (Mauriello et al., 1999).

In one such example of microencapsulation, lyophilized bacteria are suspended in 10 ml of 5% glucose saline solution in a volume so as to obtain a heavy suspension of bacteria which contains approximately 10⁹ organisms per ml, at 0° C. to 4° C. The suspension of bacteria may then be rapidly, but gently, stirred while 0.2-0.4 ml of sodium alginate solution (1.5% weight by volume) is added. The above mixture may then be transferred into a sterile container by using a nitrogen stream through a 14 gauge sheathed needle. The mixture may then be forced through a 30 gauge multi-beveled needle under pressure using a large syringe and nitrogen stream. Very small droplets are generated at the end of the needle, which are then dried by the nitrogen and air stream around the 30 gauge needle, and the droplets are collected in an aqueous solution of 1.3-2% calcium chloride where they gel. Thereafter, they are washed at least three times with 0.08-0.13% 2-(N-cyclohexyl-amino) ethanesulfonic acid (CHES) solution and 1.0-1.5% calcium chloride solution. The gelled droplets or little spheres are further washed with at least a five-fold excess of the 0.1% CHES 1.1% calcium chloride, and normal saline solution. The resultant spheres are then “snap frozen” in liquid nitrogen and then lyophilized. After these steps, the encapsulated organisms can be used in the formulations of the present invention. Other examples of microencapsulation can be found for example in U.S. Pat. No. 5,641,209 that is herein incorporated by reference.

### g. Lyophilization

Dry microorganism cultures may be prepared according to the invention, in addition to any constituents present from a fermentation medium, such as metabolic products, the medium may comprise at least one matrix material with or without other stabilizing substances. These materials are preferably selected from inorganic salts or buffers, at least one other compound which is selected from mono-, oligo- and polysaccharides, polyols, polyethers, amino acids, oligo- and polypeptides, milk-derived compounds, organic carboxylic acids, mineral compounds, organic carrier materials such as wheat semolina bran, alginates, DMSO, PVP (polyvinylpyrrolidone), CMC (carboxymethylcellulose), alpha-tocopherol, beta-carotene and mixtures thereof.

Examples of suitable saccharide carrier components are sucrose, fructose, maltose, dextrose, lactose and maltodextrin. An example of a suitable polyol is glycerol. Examples of suitable amino acids are glutamic acid, aspartic acid and the salts thereof. An example of a suitable peptide carrier is peptone. An example of a milk-derived compound is, in addition to the abovementioned maltodextrin, also sweet whey powder. Suitable organic carboxylic acids are, for example, citric acid, malic acid and L-ascorbic acid. Examples of suitable mineral carriers are montmorillonite and palygorskite.

In certain aspects of the invention mixtures of the abovementioned classes of substances may be employed. Mixtures of this type preferably comprise, as main component, a matrix material, such as one of the abovementioned saccharide components or, for example, sweet whey powder, with or without a minor content of at least one further component, such as a buffer component (for example citric acid) or an antioxidant (for example L-ascorbic acid or α.-tocopherol). The addition of further stabilizing constituents, such as sodium glutamate and/or peptone, has likewise proved to be advantageous.

The matrix component is customarily used in carrier compositions usable according to the invention in about 5 to 30 times the amount of the other carrier constituents. Examples of particularly suitable carrier combinations are: a) sweet whey powder/citric acid/L-ascorbic acid (weight ratio about 40:1:1). b) maltodextrin/lactose/citric acid/L-ascorbic acid (weight ratio about 20:20:1:1), unsupplemented or supplemented by about 1.5 parts of beta-carotene and 0.5 part of alpha-tocopherol per part of citric acid. c) maltodextrin/sodium glutamate/L-ascorbic acid (weight ratio about 10:1.5:1). d) lactose/glucose/peptone/citric acid (weight ratio about 6:6:1.2:1).

The carrier substances according to the invention can be added to the microorganism suspension either as solid or in dissolved form. However, preferably, a sterile solution of the carrier/carriers is prepared, this is cooled to a temperature of from 4° C. to 10° C. and this is mixed with the likewise cooled microorganism suspension with gentle stirring. To prepare a homogeneous suspension, the resultant mixture is stirred with further cooling for a period of from about 10 minutes to 1 hour.

The microorganism suspension containing the carrier added in the manner described above can then be dried in various ways. Suitable drying processes are in principle freeze drying, fluidized-bed drying and, preferably, spray-drying. For the purposes of the present invention, spray-drying also comprises modified spray-drying processes, such as spray-agglomeration or agglomerating spray-drying. The latter process is also known under the name FSD (fluidized spray-dryer) process.

Freeze-drying for preparing dry microorganism cultures according to the invention can be carried out, for example, on the basis of the freeze-drying process described in U.S. Pat. No. 3,897,307. The contents of these publications are hereby incorporated completely by reference.

Another, drying process contemplated for use in the present invention is spray-drying. Those methods which can be used according to the invention are essentially all spray-drying techniques known in the art. The material to be sprayed can, for example, be dried concurrently or countercurrently; spraying can be carried out by means of a single-component or multiple-component nozzle or by means of an atomizer wheel.

Preference is given according to the invention to the use of material to be sprayed having a solids content (after addition of the carrier) of from about 10 to 40, such as from about 10 to 25% by weight.

One particular factor according to the invention is the use of preconditioned, i.e. low-moisture, drying air. Preferably, use is made of compressed air having a dew point at about −25° C.

The drying process according to the invention may be carried out in such a manner that a very low residual moisture content is present in the dry material. The percentage water content is preferably from about 2 to 3% by weight. This may be achieved by adding a post-drying step subsequently to the spray-drying step. The drying material for this purpose is, for example, post-dried in a fluidized bed, preferably at a temperature in the range of from 15 to 50.degree. C., for a period of, for example, from 15 minutes to 20 hours. Again, preferably, conditioned compressed air or conditioned nitrogen serves as drying gas. However, the post-drying can also be performed by applying a vacuum of from about 1 to 50 mm Hg for a period of from about 15 minutes to 20 hours and at a temperature of from about 15 to 50° C. In this case, preference is given to stirring the drying material, for example, using a paddle agitator.

Instead of the above-described physical post-drying processes, it is also conceivable to add specific desiccants to the dry material obtained from the spray-drying. Examples of suitable desiccants are inorganic salts, such as calcium chloride and sodium carbonate, organic polymers, such as the product obtainable under the trade name Kollidion 90 F, and silicon-dioxide-containing desiccants, such as silica gel, zeolites and desiccants which are obtainable under the trade name Tixosil 38, Sipernat 22 S or Aerosil 200.

The content of viable microorganisms is in the range of from about 5×10⁸ to 1×10¹² cfu/g of dry matter. These preparations are also called according to the invention powder concentrates. Since, for individual final applications, lower contents of viable microorganisms are also completely sufficient, powder concentrates of this type can therefore if appropriate be blended to the final count of viable microorganisms by mixing with further inert carrier material.

### h. Uses of Formulated Bacterial Products

The methods and formulations of the present disclosure can be adjusted to provide beneficial effects to many types of animals, including ruminal fermentors, cecal fermentor and intestinal fermentors. In one preferred embodiment, the product is fed to ruminal fermentors to reduce scours events, improve animal health and animal productivity. Ruminal fermentors that might benefit from the present invention include but are not limited to: cattle, sheep, goats, camels, llama, bison, buffalo, deer, wildebeest, antelope, and any other pre-gastric fermentor. In another embodiment, the product is fed to cecal fermentors to reduce scours events, improve animal health and animal productivity. Cecal fermentors that might benefit from the present invention include but are not limited to: horses, ponies, elephants, rabbits, hamsters, rats, hyraxes, guinea pigs, and any other post-gastric fermentor that using the cecum as the primary location of fermentative digestion. In another embodiment, product is fed to intestinal fermentors to reduce scours events, improve animal health and animal productivity. Intestinal fermentors that might benefit from said invention include but are not limited to: humans, pigs, chickens, and other post-gastric fermentor using the large intestine as the primary location of fermentative digestion.

The amount of microorganism administered to the animal feed can be any amount sufficient to achieve the desired increase in animal efficiency and/or animal health. This amount can be anywhere from 1 to 10¹³ organisms per kg of animal feed. For example, amounts of about 10⁴ cfu/gram feed, about 5×10⁴ cfu/gram feed, about 10⁵ cfu/gram feed, about 5×10⁵ cfu/gram feed, or ranges between 1 to 10¹³ organisms per kg of animal feed can be used. In some embodiments, the dried biological may be administered to an animal through a variety of means including, but not limited to, being distributed in an aqueous solution and subsequently being applied to animal feed, water source, or directly fed to the animal, or through direct application of the product onto animal feed or direct administration or consumption by the animal.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit or scope of the invention. The following Examples are offered by way of illustration and not by way of limitation.

### Example 1

### Preparation of Reagents

### a. Lactobacilli MRS Agar And Broth

Lactobacilli MRS agar and broth are recommended for the use in the isolation of Lactobacillus species. Lactobacilli MRS agar and broth are based on the formulations of de Man et al., J. Appl. Bacteriol., 23:130, 1960. Difco™ & BBL™ Manual, 2nd Edition. The agar and broth were demonstrated by de Man et al., to support Lactobacilli growth from oral, fecal dairy and other sources. Lactobacilli MRS Agar and broth contain peptone and dextrose, both of which supply nitrogen, carbon and other elements necessary for growth. Polysorbate 80, acetate, magnesium and manganese provide growth factors for culturing a variety of lactobacilli.

In brief, to generate Lactobacilli MRS Agar, into one liter of distilled water: 10.0 g proteose peptone No. 3, 10.0 g beef extract, 5.0 g yeast extract, 20.0 g dextrose, 1.0 g polysorbate 80, 2.0 g ammonium citrate, 5.0 g sodium acetate, 0.1 g magnesium sulfate, 0.05 g manganese sulfate, 2.0 g dipotassium phosphate and 15.0 g agar. Lactobacilli MRS broth is generated by the same methods without the addition of agar. These materials are readily obtained from Becton Dickinson and Company, Franklin Lakes N.J.

### b. Lactobacilli Fermentation Medium

Lactobacilli fermentation medium may be made by adding into 450 ml of distilled water the following ingredients: 4.0 g trypticase, 3.0 g casamino acids, 6.0 g yeast extract, 0.5 g sodium acetate trihydrate, 1.0 g ammonium citrate, 1.0 g potassium phosphate, 1.0 g magnesium sulfate, 0.05 g manganese sulfate and 500 μl polyoxyethylene (20) sorbitan monooleate.

### c. LBS (Lactobacillus Selection) Medium

LBS medium may be made by adding into 1 L of distilled water the following ingredients: 10.0 g trypticase, 5.0 g yeast extract, 25.0 g sodium acetate hydrate, 20.0 g glucose, 2.0 g ammonium citrate, 6.0 g monopotassium phosphate, 0.575 g magnesium sulfate anhydrous, 0.12 g manganese sulfate monohydrate, 0.034 g ferrous sulfate, and 1 ml polyoxyethylene (20) sorbitan monooleate. LBS agar may be prepared by adding 15 g of agar to 1 L of the LBS medium.

### Example 2

### Stability of Dried Bacteria In FOS Carrier Incubated Under Ambient Conditions

0.24 g of the Lactobacillus freeze-dry was placed into 150 g of each pre-chilled carrier. Carriers evaluated were FOS, FOS-flow, and lactose. Products were placed into plastic jars and allowed to incubate at room temp (22° C.). At regular time intervals, 10 g of each product was suspended into 96 ml sterile PBS and allowed to dissolve at room temperature for 10 minutes. Suspensions were serially diluted in NPCl+glucose and the last three dilutions tubes plated onto NPCl+glucose plates. Plates were incubated overnight at 37° C. and colonies were counted.

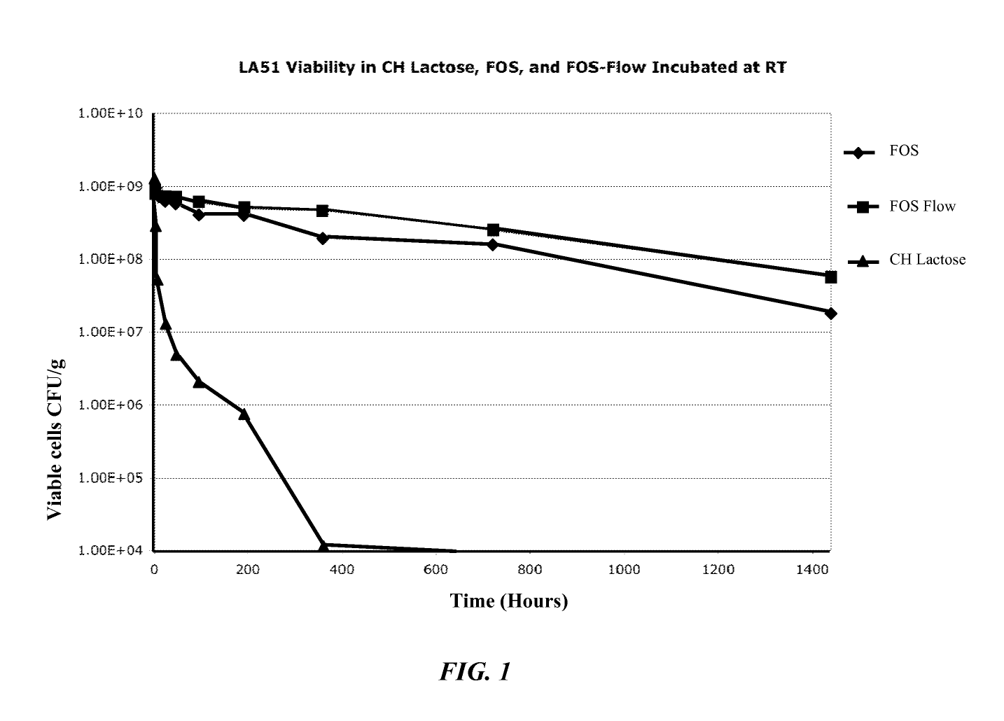

The results presented in FIG. 1 and Table 1 clearly demonstrate that freeze dried bacteria stored in FOS or FOS-Flow retain a much greater level of viability when stored at 22° C. (72° F.). After just 24 hours of storage at room temp, greater than 50% of the initial bacteria stored in FOS remain in a viable state, whereas only 1% of the bacteria are viable when stored in lactose under identical conditions.

TABLE 1
Hours

| Carrier | 0 | 4 | 8 | 24 | 48 | 96 | 192 | 360 | 720 | 1440 |
|---|---|---|---|---|---|---|---|---|---|---|
| FOS | 1.19E+09 | 8.10E+08 | 7.30E+08 | 6.50E+08 | 5.96E+08 | 4.18E+08 | 4.07E+08 | 1.95E+08 | 1.66E+08 | 1.84E+07 |
| (% | 100.0% | 68.1% | 61.3% | 54.6% | 50.1% | 35.1% | 34.2% | 16.4% | 13.9% | 1.5% |
| initial) | | | | | | | | | | |
| FOS | 9.70E+08 | 7.80E+08 | 8.60E+08 | 7.20E+08 | 6.90E+08 | 6.11E+08 | 5.01E+08 | 4.74E+08 | 2.56E+08 | 5.94E+07 |
| Flow | | | | | | | | | | |
| (% | 100.0% | 80.4% | 88.7% | 74.2% | 71.1% | 63.0% | 51.6% | 48.9% | 26.4% | 6.1% |
| initial) | | | | | | | | | | |
| CH | 1.21E+09 | 3.04E+08 | 5.50E+07 | 1.30E+07 | 4.96E+06 | 2.14E+06 | 7.70E+05 | 1.21E+04 | 9.30E+03 | 1.00E+00 |
| Lactose | | | | | | | | | | |
| (% | 100.0% | 25.1% | 4.5% | 1.1% | 0.41% | 0.18% | 0.064% | 0.0010% | 0.0008% | 0.0% |
| initial) | | | | | | | | | | |

### Example 3

Stability of dried bacteria in FOS carrier incubated at 37° C.

0.24 g of the Lactobacillus freeze-dry was placed into 150 g of each pre-chilled carrier. Carriers evaluated were FOS, FOS-Flow, and lactose. Products were placed into plastic jars and allowed to incubate at 37° C. At regular time intervals, 10 g of each product was suspended into 96 ml sterile PBS and allowed to dissolve at room temperature for 10 minutes. Suspensions were serially diluted in NPCl+glucose and the last three dilutions tubes plated onto NPC+glucose plates. Plates were incubated overnight at 37° C. and colonies counted.

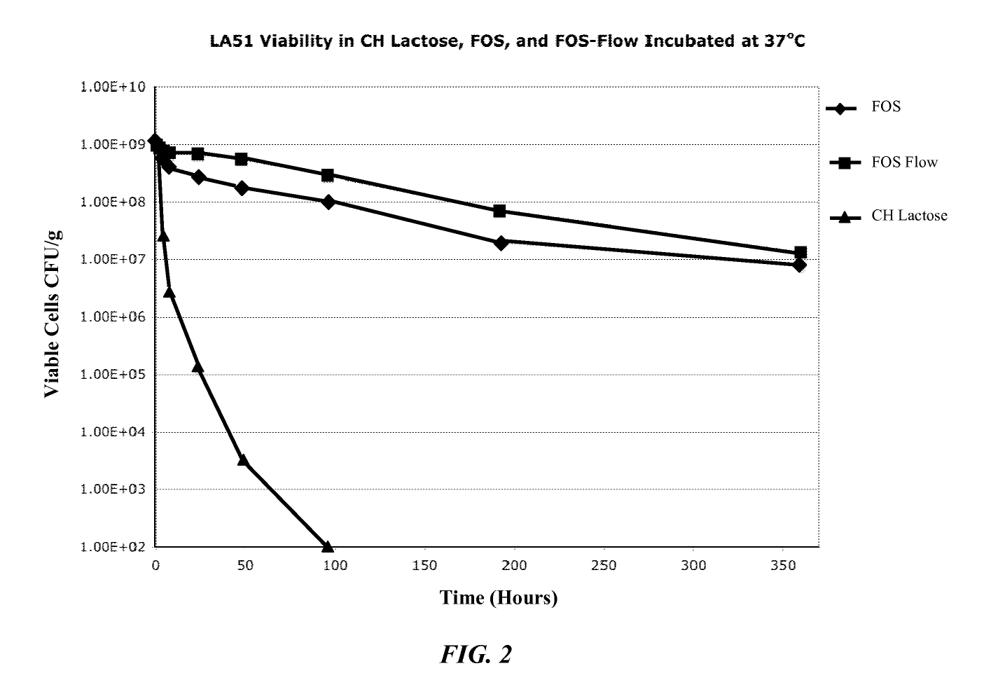

Higher temperatures exacerbate loss of viability in freeze-dried microorganisms. Such conditions can be experienced during processing, packaging, shipment, and storage of the products. The results presented in FIG. 2 and Table 2 clearly demonstrate that freeze dried bacteria stored in FOS or FOS-Flow retain a much greater level of viability when stored at 37° C. Similar viability trends were seen in products incubated at 22° C., but the rate of loss was greater in all carriers than when stored at room temperature. Within 8 hours of exposure at 37° C., >99% of the freeze-dried Lactobacillus were dead in the lactose carrier. Whereas, there was still 35% and 71% retention of viable cells in the FOS and FOS-flow carriers, respectively, with the same time of exposure. After 192 hours (8 days) of exposure at 37° C., the FOS retained 1.7% of viable cells and the FOS-flow still contained 7.1% of the initial population. No viable cells were found in the lactose carrier.

TABLE 2
Hours

| Carrier | 0 | 2 | 4 | 8 | 24 | 48 | 96 | 192 | 360 |
|---|---|---|---|---|---|---|---|---|---|
| FOS | 1.18E+09 | 7.72E+08 | 5.72E+08 | 4.20E+08 | 2.68E+08 | 1.78E+08 | 1.02E+08 | 2.02E+07 | 8.06E+06 |
| (% initial) | 100.0% | 65.4% | 48.5% | 35.6% | 22.7% | 15.1% | 8.7% | 1.7% | 0.7% |
| FOS Flow | 1.00E+09 | 8.58E+08 | 8.04E+08 | 7.12E+08 | 6.92E+08 | 5.70E+08 | 2.92E+08 | 7.08E+07 | 1.30E+07 |
| (% initial) | 100.0% | 85.5% | 80.1% | 70.9% | 68.9% | 56.8% | 29.1% | 7.1% | 1.3% |
| CH | 1.03E+09 | 5.90E+08 | 2.60E+07 | 3.00E+06 | 1.34E+05 | 3.40E+03 | 1.00E+02 | 1.00E+00 | 1.00E+00 |
| Lactose | | | | | | | | | |
| (% initial) | 100.0% | 57.3% | 2.5% | 0.29% | 0.013% | 0.00033% | 0.00001% | 0.00% | 0.00% |

### Example 4

### Ability of Microorganisms To Utilize FOS And Lactose

A series of different tubes of media were amended with variable amounts of either lactose or FOS from 20% stock solutions. Four sets of these tubes were prepared and inoculated with 50 μl of a 1:10 diluted overnight culture of each organism. The tubes were incubated overnight at 37° C. Culture optical density was measured at a wavelength of 600 nm. Final pH of the media was also recorded.

Increases in culture optical density indicates that an organism is capable of utilizing the provided carbon source. However, not all organisms, like E. coli, Salmonella, and Listeria, require a carbohydrate carbon source for growth and are able to use amino acids and vitamins provided in the medium from complex medium ingredients such as yeast extract, casamino acids, and trypticase. This results in no increase or small increases in culture optical density.

However, another indicator that the organisms are capable of using the provided carbon source is to monitor change in pH of the medium after bacterial growth. Typically, when carbohydrate carbon sources are utilized by an organism, waste byproducts include acids that are released into the surrounding medium. The release of these acids results in a reduction of the pH of the growth medium. Thus although there may be no large increase in culture optical density in the presence of a carbon source, a reduction in the growth medium pH in the same tube indicates that the organisms can utilize it. If there is no increase in culture optical density or reduction in growth medium pH, then the organisms cannot readily use the provided carbon source.

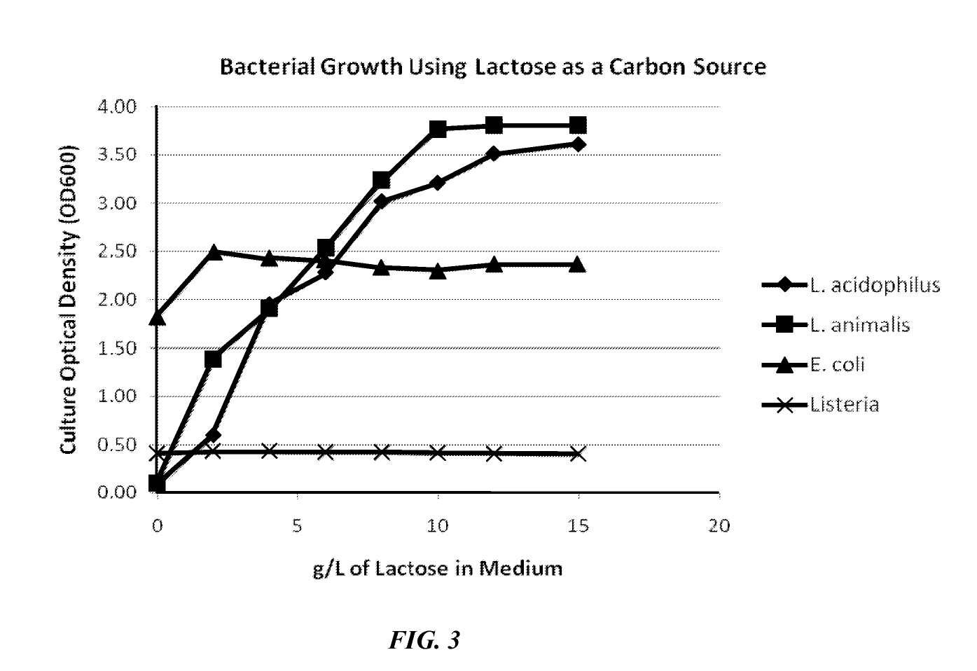

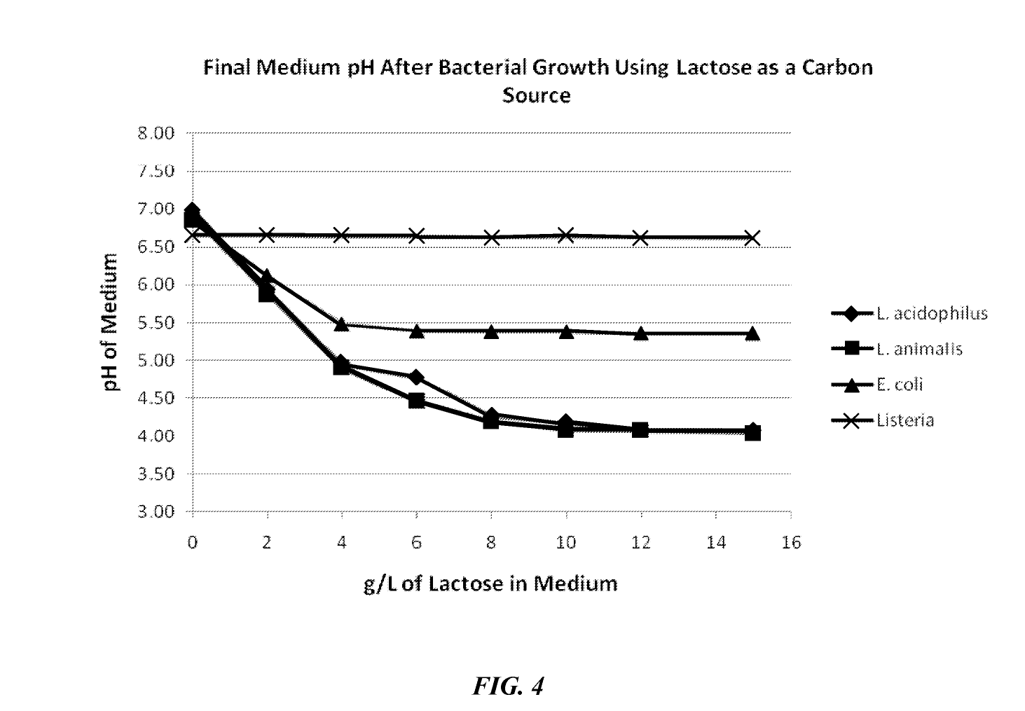

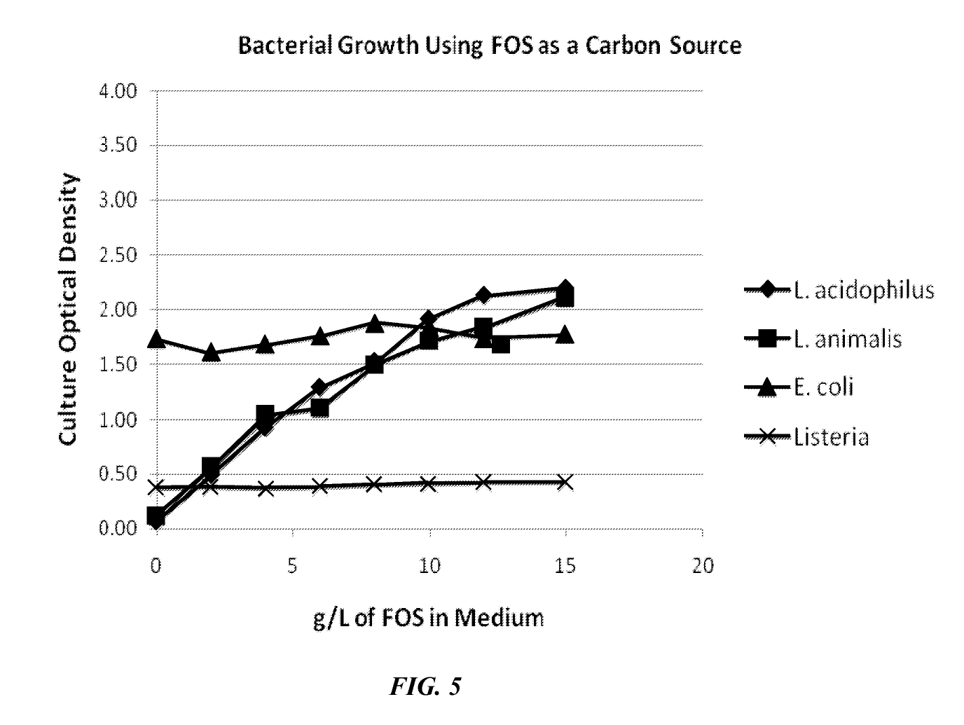

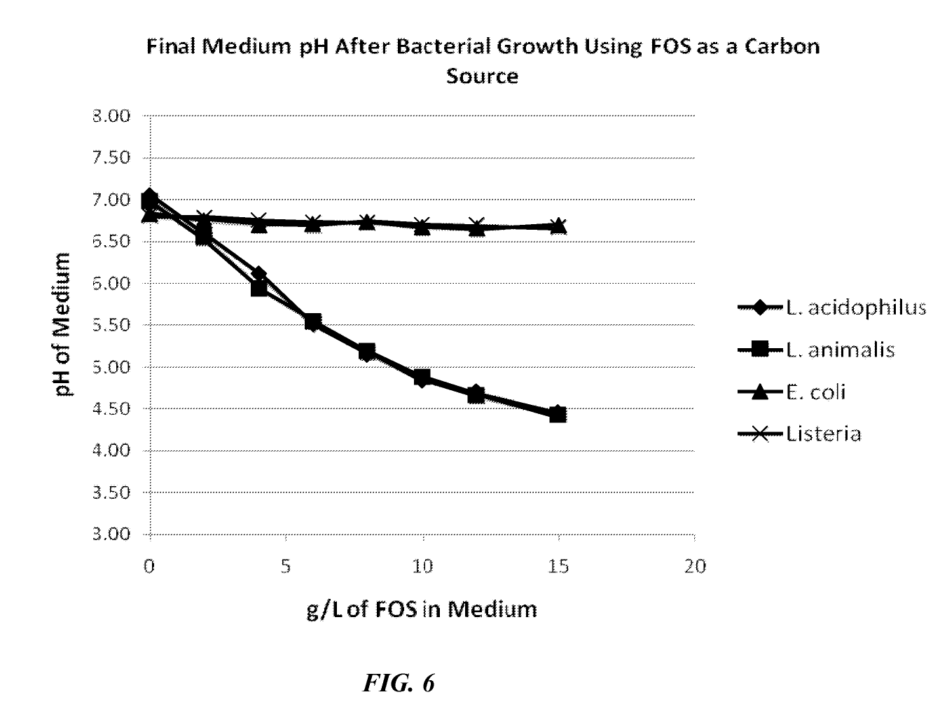

Shown in FIG. 3 and FIG. 4 is the data indicating that E. coli, L. acidophilus, and L. animalis can use lactose as a carbon source because of the increases in culture optical density and concurrent reduction in growth medium pH. This demonstrates that Listeria cannot use lactose for growth with the absence of increased culture optical density or decreased medium pH. However, FIG. 5, FIG. 6 and Table 3 indicate that Lactobacillus species can readily utilize the provided FOS, whereas neither the potential pathogens E. coli or Listeria can utilize the provided FOS for growth.

### Example 5

### Ability of Lactic Acid Bacteria to Kill E. coli Using FOS for Growth

Tubes containing an MRS medium without glucose were amended with variable amounts of sterile-filtered glucose (20 g/L) or sterile-filtered FOS (20 g/L). Approximately 1×10⁴ viable cells (based culture optical density) of a single strain of E. coli and either the Lactobacillus strains MBLA1, MBLA2, and MBLA3 were added to the test tubes. Tubes were incubated for 6 or 12 hours at 37° C. After incubation, tubes were serially diluted and 100 μl spread onto Luria-Bertani plates to enumerate viable E. coli. The percent E. coli inhibition was calculated as concentration of viable E. coli cells in tubes co-incubated with Lactobacillus strains divided by the concentration of viable E. coli incubated without Lactobacillus multiplied by 100.

The lactobacilli investigated here were able to reduce the growth of E. coli whether using either glucose or FOS as a carbon source. After six hours of co-incubation both MBLA1 and MBLA3 showed greater than a 70% reduction in viable E. coli compared with the control of E. coli grown alone. MBLA2 reduced E. coli growth by 53%. Each of the strains reduced E. coli growth with FOS, but slightly less inhibition as was seen in the presence of glucose. After 12 hours of co-incubation, dramatic reductions in viable E. coli were seen with 99.1%, 98.4%, and 99.8% reduction for MBLA1, MBLA2, and MBLA3, respectively. After 6 hours of incubation with FOS as a carbon source, MBLA1, MBLA2, and MBLA3 reduced the pH of the medium to 4.84, 5.35, and 4.71, respectively. After 12 hours the pH further decreased to 4.31, 4.25, and 4.25, respectively. These results demonstrate the ability of the FOS to select for natural Lactobacillus populations capable of inhibiting and even killing potential intestinal pathogens.

TABLE 4

| Carbon | Incubation | Lactobacillus Strain | | |
|---|---|---|---|---|
| Source | Time | MBLA1 | MBLA2 | MBLA3 |
| FOS | 12 hr | 99.1% | 98.4% | 99.8% |
| FOS | 6 hr | 44.4% | 34.8% | 73.3% |
| Glucose | 6 hr | 69.9% | 46.7% | 78.3% |

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

U.S. Pat. App. Pub. No. 20060039899

U.S. Pat. No. 3,897,307

U.S. Pat. No. 4,873,229

U.S. Pat. No. 4,902,674

U.S. Pat. No. 4,987,124

U.S. Pat. No. 5,032,579

U.S. Pat. No. 5,529,793

U.S. Pat. No. 5,534,271

U.S. Pat. No. 5,641,209

U.S. Pat. No. 5,733,774

U.S. Pat. No. 5,785,990

U.S. Pat. No. 6,241,983

U.S. Pat. No. 6,368,591

U.S. Pat. No. 6,783,780

U.S. Pat. No. 6,960,341

U.S. Pat. No. 7,001,624

U.S. Pat. No. 7,063,836

U.S. Pat. No. 7,101,565

Collins and Gibson, Am. J. Clin. Nutrition, 69: 1052S-1057S, 1999.

Gibbs et al., Int. J. Food Sci. Nutr., 50: 213-224, 1999.

Gibson and Roberfroid, J. of Nutrition, 125: 1401-1412, 1995.

Mauriello et al., J. Food Prot., 62: 773-777, 1999.

O'Riordan et al., J. Appl. Microbiol., 91:1059-1066, 2001.

Roberfroid, Am. J. Clin. Nutrition, 71: 1682S-1687S, 2000.

Schultz et al., J. of Pediatric Gastroenterology and Nutrition, 38:293-297, 2004.

We claim:

1. A formulation comprising:
i) at least one species or strain of probiotic microorganism, and
ii) fructo-oligosaccharide;
wherein the fructo-oligosaccharide itself comprises 50% or greater by weight of the formulation; and
wherein the formulation is freeze-dried.

2. The formulation of claim 1, wherein the probiotic microorganism is Enterococcus faecium, Bacillus licheniformis, Lactococcus lactis, Bacillus subtilis, Bifidobacterium adolescentis, Bifidobacterium animalis, Bifidobacterium bifidum, Bifidobacterium infantis, Bifidobacterium longum, Bifidobacterium thermophilum, Lactobacillus acidophilus, Lactobacillus agilis, Lactobacillus alactosus, Lactobacillus alimentarius, Lactobacillus amylophilus, Lactobacillus amylovorans, Lactobacillus amylovorus, Lactobacillus animalis, Lactobacillus batatas, Lactobacillus bavaricus, Lactobacillus bifermentans, Lactobacillus bifidus, Lactobacillus brevis, Lactobacillus buchnerii, Lactobacillus bulgaricus, Lactobacillus catenaforme, Lactobacillus casei, Lactobacillus cellobiosus, Lactobacillus collinoides, Lactobacillus confusus, Lactobacillus coprophilus, Lactobacillus coryniformis, Lactobacillus corynoides, Lactobacillus crispatus, Lactobacillus curvatus, Lactobacillus delbrueckii, Lactobacillus desidiosus, Lactobacillus divergens, Lactobacillus enterii, Lactobacillus farciminis, Lactobacillus fermentum, Lactobacillus (rigidus, Lactobacillus fructivorans, Lactobacillus fructosus, Lactobacillus gasseri, Lactobacillus halotolerans, Lactobacillus helveticus, Lactobacillus heterohiochii, Lactobacillus hilgardii, Lactobacillus hordniae, Lactobacillus inulinus, Lactobacillus jensenii, Lactobacillus jugurti, Lactobacillus kandleri, Lactobacillus kefir, Lactobacillus lactis, Lactobacillus leichmannii, Lactobacillus lindneri, Lactobacillus malefermentans, Lactobacillus mali, Lactobacillus maltaromicus, Lactobacillus minor, Lactobacillus minutus, Lactobacillus mobilis, Lactobacillus murinus, Lactobacillus mucosae, Lactobacillus pentosus, Lactobacillus plantarum, Lactobacillus pseudoplantarum, Lactobacillus reuteri, Lactobacillus rhamnosus, Lactobacillus rogosae, Lactobacillus tolerans, Lactobacillus torquens, Lactobacillus ruminis, Lactobacillus sake, Lactobacillus salivarius, Lactobacillus sanfrancisco, Lactobacillus sharpeae, Lactobacillus trichodes, Lactobacillus vaccinostercus, Lactobacillus viridescens, Lactobacillus vitulinus, Lactobacillus xylosus, Lactobacillus yamanashiensis, Lactobacillus zeae, Pediococcus acidilactici, Pediococcus pentosaceus, Streptococcus cremoris, Streptococcus diacetylactis, Streptococcus faecium, Streptococcus intermedius, Streptococcus lactis, Streptococcus thermophilus, Propionibacterium spp., Propionibacterium freudenreichii, Propionibacterium acidipropionici, Propionibacterium jensenii, Propionibacterium thoenii, Propionibacterium cyclohexanicum, Propionibacterium granulosum, Propionibacterium microaerophilum, Propionibacterium propionicum, Propionibacterium acnes, Propionibacterium australiense, Propionibacterium avidum or strains or combinations thereof.

3. The formulation of claim 1, wherein the probiotic microorganism is a lactic-acid producing microorganism.

4. The formulation of claim 3, wherein the lactic-acid producing microorganism is Lactobacillus acidophilus, Lactobacillus agilis, Lactobacillus alactosus, Lactobacillus alimentarius, Lactobacillus amylophilus, Lactobacillus amylovorans, Lactobacillus amylovorus, Lactobacillus animalis, Lactobacillus batatas, Lactobacillus bavaricus, Lactobacillus bifermentans, Lactobacillus bifidus, Lactobacillus brevis, Lactobacillus buchnerii, Lactobacillus bulgaricus, Lactobacillus catenaforme, Lactobacillus casei, Lactobacillus cellobiosus, Lactobacillus collinoides, Lactobacillus confusus, Lactobacillus coprophilus, Lactobacillus coryniformis, Lactobacillus corynoides, Lactobacillus crispatus, Lactobacillus curvatus, Lactobacillus delbrueckii, Lactobacillus desidiosus, Lactobacillus divergens, Lactobacillus enterii, Lactobacillus farciminis, Lactobacillus fermentum, Lactobacillus frigidus, Lactobacillus fructivorans, Lactobacillus fructosus, Lactobacillus gasseri, Lactobacillus halotolerans, Lactobacillus helveticus, Lactobacillus heterohiochii, Lactobacillus hilgardii, Lactobacillus hordniae, Lactobacillus inulinus, Lactobacillus jensenii, Lactobacillus jugurti, Lactobacillus kandleri, Lactobacillus kefir, Lactobacillus lactis, Lactobacillus leichmannii, Lactobacillus lindneri, Lactobacillus malefermentans, Lactobacillus mali, Lactobacillus maltaromicus, Lactobacillus minor, Lactobacillus minutus, Lactobacillus mobilis, Lactobacillus murinus, Lactobacillus pentosus, Lactobacillus plantarum, Lactobacillus pseudoplantarum, Lactobacillus reuteri, Lactobacillus rhamnosus, Lactobacillus rogosae, Lactobacillus tolerans, Lactobacillus torquens, Lactobacillus ruminis, Lactobacillus sake, Lactobacillus salivarius, Lactobacillus sanfrancisco, Lactobacillus sharpeae, Lactobacillus trichodes, Lactobacillus vaccinostercus, Lactobacillus viridescens, Lactobacillus vitulinus, Lactobacillus xylosus, Lactobacillus yamanashiensis, Lactobacillus zeae, Lactobacillus strain MRL1, Lactobacillus strain M35, Lactobacillus strain LA45, Lactobacillus strain LA51, Lactobacillus strain L411, Lactobacillus strain NPC 747, Lactobacillus strain NPC 750, Lactobacillus strain D3, or Lactobacillus strain L7 or a combination thereof.

5. The formulation of claim 1, wherein the probiotic microorganism is a propionic acid-producing microorganism.

6. The formulation of claim 5, wherein the propionic acid-producing microorganism is Propionibacterium strain PF24, P5, P63, Pl, or MRP1 or a combination thereof.

7. The formulation of claim 1, further comprising a flow agent.

8. The formulation of claim 7, wherein the flow agent is calcium stearate, silicon dioxide or a combination thereof.

9. The formulation of claim 8, wherein the flow agent is calcium stearate.

10. The formulation of claim 7, wherein the flow agent is about 2% of the composition.

11. The formulation of claim 1, wherein the at least one species or strain of probiotic microorganism retains at least 40% viability when stored for 48 hours at 22° C.

12. The formulation of claim 11, wherein the at least one species or strain of probiotic microorganism retains at least 15% viability when stored for 48 hours at 37° C.

13. The formulation of claim 7, wherein the at least one species or strain of probiotic microorganism retains at least 60% viability when stored for 48 hours at 22° C.

14. The formulation of claim 13, wherein the at least one species or strain of probiotic microorganism retains at least 50% viability when stored for 48 hours at 37° C.
